(19) United States
(12) Patent Application Publication
(10) Pub. No.: US 2023/0131878 A1
(43) Pub. Date: Apr. 27, 2023

(54) ADDITIVELY MANUFACTURED MEDICAL IMPLANTS, METHODS FOR FORMING SAME, AND ZIRCONIUM ALLOY POWDER FOR FORMING SAME

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventor: Vivek D. Pawar, Germantown, TN (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 17/917,961

(22) PCT Filed: Apr. 14, 2021
(86) PCT No.: PCT/US2021/027272

§ 371 Date: Oct. 10, 2022

Related U.S. Application Data
(60) Provisional application No. 63/014,830, filed on Apr. 24, 2020.

(51) Int. Cl. A61F 2/30; B22F 10/28; B22F 10/62; B33Y 10/00; B33Y 40/20; B33Y 80/00; B33Y 70/10; B22F 1/16

(52) U.S. Cl. CPC A61F 2/3094; B22F 10/28; B22F 10/62; B33Y 10/00; B33Y 40/20; B33Y 80/00; B33Y 70/10; B22F 1/16; A61F 2310/00089; A61F 2002/30968; A61F 2002/30971; A61F 2002/3097; B22F 2301/205; B22F 2302/25; B22F 2304/10; B22F 2998/10

(57) ABSTRACT

The present disclosure provides zirconium powder particles comprising pure zirconium powder particles with an oxide layer ranging from 0.05 to 5 microns in thickness and/or zirconium alloy powder particles with an oxide layer ranging from 0.05 to 5 microns in thickness. In some embodiments, the zirconium powder particles may be spherical particles, the zirconium powder particles may range from 5 microns to 125 microns in diameter, and/or the zirconium powder particles may have a median particle size ranging from 25 to 70 microns in diameter. The present disclosure further provides methods of producing medical implants or medical implant components by a process that comprises selectively applying energy to such zirconium powder particles to build the medical implants or the medical implant components. In some embodiments, the methods comprise repeatedly forming a layer of zirconium powder particles and irradiating the layer of zirconium powder particles with an energy source.

### CROSS-REFERENCE TO RELATED APPLICATIONS

This is a non-provisional of, and claims the benefit of the filing date of, pending U.S. provisional patent application No. 63/014,830, filed Apr. 24, 2020, entitled “Methods for Additive Manufacturing of Zirconium Alloys and a Zirconium Alloy Powder” the entirety of which application is incorporated by reference herein.

### FIELD OF THE DISCLOSURE

The present disclosure is directed to medical implants, including orthopedic implants. More particularly the present disclosure is directed to additively manufactured medical implants, to additive manufacturing methods for forming medical implants, and to zirconium alloy powder particles for forming medical implants.

### BACKGROUND

Additive manufacturing techniques include those known in the art such as solid free-form fabrication (SFF), selective laser sintering (SLS), direct metal fabrication (DMF), direct metal laser sintering (DMLS), electron beam melting (EBM), and selective laser melting (SLM), among others. Additive manufacturing methods allow for three-dimensional structures to be constructed one layer at a time from a powder which is solidified by irradiating a layer of the powder with an energy source such as a laser or an electron beam.

Currently, a majority of the focus of additive manufacturing technology in medical devices is for titanium, cobalt, and stainless steel alloys. One of the key requirements for these technologies is the material powder size. Typical median powder size is 25-70 micron in diameter and spherical in shape. The smaller size ensures localized melting (better feature resolution) and spherical shape ensures smooth flow of the material as each layer is built. Because of the reactive nature of titanium, zirconium and their alloys, it is desirable to produce powder that is stable and will not combust or explode when exposed to an energy source in presence of a small amount of oxygen. Although this has been achieved for titanium, it is difficult to do the same for zirconium, as zirconium is much more reactive than titanium. Another challenge, which is specific to electron beam processing is “caking” of the powder during the build. Because electron beam processing uses elevated temperature during the additive manufacturing process, the powder particles tend to fuse forming a “cake” around the actual product. This caked powder needs to be removed and sieved after the process is completed for powder reuse.

It is with this in mind that the present disclosure is provided.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

The present disclosure provides zirconium powder particles that have been oxidized in a controlled manner. This can be accomplished either during manufacturing of the zirconium powder particles or after the zirconium powder particles are produced. This controlled oxidation will form a small but significant amount of oxide on the surface and thus will prevent further oxygen pick-up from the powder during transport and usage in additive manufacturing. Because the oxide is relatively thin, overall heating and melting of the powder should not be significantly affected during the additive manufacturing process. The oxide on the surface will also reduce combustibility and prevent caking, predominantly from surface bonding due to high temperature in electron beam additive manufacturing processing. In this regard, although zirconium powder particles previously used in additive manufacturing have a native oxide coating, this coating is very thin (less than 50 Angstroms (0.005 micron)) and thus has a minimal impact on various properties of the material, including combustibility and caking.

In some embodiments, the present disclosure provides oxidized zirconium powder particles that comprise (a) pure zirconium powder particles with an oxide layer ranging from 0.05 to 5 microns in thickness, for example, ranging from 0.05 to 0.1 to 0.25 to 0.5 to 1 to 2.5 to 5 microns in thickness (i.e., between any two of the preceding values), more typically from 0.05 to 1 micron in thickness, and/or (b) zirconium alloy powder particles with an oxide layer ranging from 0.05 to 5 microns in thickness, for example, ranging from 0.05 to 0.1 to 0.25 to 0.5 to 1 to 2.5 to 5 microns in thickness, more typically from 0.05 to 1 micron in thickness.

In some embodiments, the oxidized zirconium powder particles are spherical particles.

In some embodiments, at least 95 wt %, more typically at least 99 wt %, of the oxidized zirconium powder particles are between 5 microns and 125 microns in diameter, more typically, between 25 and 125 microns.

In some embodiments, the oxidized zirconium powder particles have a median particle size that is between 25 and 70 microns.

In some embodiments, oxidized zirconium powder particles such as those described above may be formed by a plasma rotating electrode process in which, instead of an inert atmosphere in the plasma chamber, controlled amounts of oxygen are be introduced.

In some embodiments, oxidized zirconium powder particles such as those described above may be formed on previously produced pure zirconium powder particles or previously produced zirconium alloy powder particles, for example, by employing oxidation processes such as air oxidation at elevated temperatures, steam oxidation, water oxidation or oxidation in a salt bath.

In some embodiments, the present disclosure provides a method of producing a medical implant or a medical implant component by a process that comprises selectively applying energy to a zirconium powder comprising oxidized zirconium powder particles in accordance with any of the above embodiments, thereby building the medical implant or the medical implant component.

In some embodiments, the zirconium powder further comprises unoxidized zirconium powder particles that comprise (a) unoxidized pure zirconium powder particles and/or (b) unoxidized zirconium alloy powder particles.

In some embodiments, the unoxidized zirconium powder particles have a median particle size that is the same as or greater than a median particle size of the oxidized zirconium powder particles. In some of these embodiments, the unoxidized zirconium powder particles may have a median particle size that ranges from 1 to 5 times (e.g., from 1 to 2 to 3 to 4 to 5 times) a median particle size of the oxidized zirconium powder particles.

In some embodiments, a weight ratio of the oxidized zirconium powder particles to the unoxidized zirconium powder particles may range from 0.5:1 to 2:1.

In some embodiments, which can be used in conjunction with any of the above embodiments, the medical implant or medical implant component is produced in a layer-wise fashion by dispensing and irradiating the zirconium powder particles in preselected areas one layer at a time.

In some embodiments, which can be used in conjunction with any of the above embodiments, the method of producing the medical implant or medical implant component comprises repeatedly forming a layer of the zirconium powder and irradiating the layer of zirconium powder with an energy source to melt, fuse and/or sinter the zirconium powder particles until the medical implant or medical implant component is formed.

In some embodiments, which can be used in conjunction with any of the above embodiments, the energy is applied to the zirconium powder by irradiating the zirconium powder in predetermined areas with a laser beam or an electron beam.

In some embodiments, which can be used in conjunction with any of the above embodiments, a laser beam or an electron beam is scanned over a first layer of powder particles in a first direction, after which a further layer of metallic powder particles is provided over the first layer, and a laser beam or an electron beam is scanned over the further layer of metallic powder particles in second direction that is transverse to the first direction.

In some embodiments, which can be used in conjunction with any of the above embodiments, the energy is applied to the zirconium powder in a vacuum and at temperatures ranging from 500 degrees F. to 1300 degrees F.

In some embodiments, which can be used in conjunction with any of the above embodiments, the method further comprises oxidizing at least one surface of the medical implant or medical implant component that is formed to form a ceramic surface. For example, a ceramic surface comprising an oxide and a diffusion hardened zone may be formed. Such a surface may function as a wear-resistant surface on articulating surfaces of medical implants including orthopedic implants.

In some embodiments, the present disclosure pertains to a medical implant or medical implant component that is formed from a method in accordance with any of the above embodiments.

In some embodiments, the medical implant or medical implant component is substantially nonporous.

In some embodiments, the medical implant or medical implant component has one or more substantially nonporous regions and one or more substantially porous regions.

In some embodiments the medical implant or medical implant component is a hip implant, a knee implant, a shoulder implant, an ankle implant, a spinal implant, or a component thereof.

Embodiments of the present disclosure provide numerous advantages. For example, in accordance with some embodiments of the present disclosure, zirconium alloy powder can be prepared in a desirable size range that is stable when used in additive manufacturing processes based on either laser or e-beam processing.

As another example, zirconium alloy powder can be prepared in a desirable size range that is resistant to caking when used in additive manufacturing processes based e-beam processing.

Further features and advantages of at least some of the embodiments of the present invention, as well as the structure and operation of various embodiments of the present invention, are described in detail below with reference to the accompanying drawings.

### BRIEF DESCRIPTION OF THE DRAWINGS

By way of example, a specific embodiment of the disclosed device will now be described, with reference to the accompanying drawings, in which:

FIG. 1 illustrates an e-beam machine for producing an additively manufactured part.

FIG. 2 illustrates a collection of zirconium powder.

FIG. 3 illustrates a close-up of one of the zirconium particles in FIG. 1.

FIG. 4 illustrates a powder bed arrangement of the current invention with varying size particles.

The drawings are not necessarily to scale. The drawings are merely representations, not intended to portray specific parameters of the disclosure. The drawings are intended to depict example embodiments of the disclosure, and therefore are not be considered as limiting in scope. In the drawings, like numbering represents like elements.

Furthermore, certain elements in some of the figures may be omitted, or illustrated not-to-scale, for illustrative clarity. The cross-sectional views may be in the form of “slices”, or “near-sighted” cross-sectional views, omitting certain background lines otherwise visible in a “true” cross-sectional view, for illustrative clarity. Furthermore, for clarity, some reference numbers may be omitted in certain drawings.

### DETAILED DESCRIPTION

The following description of the depicted embodiment(s) is merely exemplary in nature and is in no way intended to limit the invention, its application, or uses.

As used herein, “pure zirconium metal” refers to compositions that contain at least 99% w/w percent zirconium.

As used herein, “zirconium alloy” includes alloys having a least 5% (w/w) zirconium, typically greater than 70% w/w zirconium and less than 99% w/w percent zirconium. In some embodiments, the zirconium alloy comprises zirconium and one or more of niobium, titanium, tantalum or hafnium. In a particular embodiment, the zirconium alloy is Zr-2.5Nb, among other possibilities. The alloys can be polycrystalline or amorphous or single crystals or combinations of same.

As used herein “unoxidized” powder particles are particles that do not have any oxide coating, other than a native oxide layer that may be present on such particles.

As used herein, the term “vacuum” refers to a pressure of less than about 10⁻² torr.

As used herein, “additive manufacturing” refers to methods used to build objects, including but not limited to powder bed fusion, direct metal laser sintering, e-beam processing including electron beam melting, fused deposition modelling, laser engineered net shaping, solid free-form fabrication, selective laser sintering, direct metal fabrication, and selective laser melting.

There is provided a method and a product resulting thereof. The method includes providing zirconium powder, for example, a pure zirconium metal or zirconium alloy (e.g., Zr-2.5Nb alloy) powder particles, with an average particle diameter in the range of 5-125 microns, more typically 25-125 microns and oxidizing the surface to a depth of less than 5 microns, typically 0.05-5 microns, more typically 1-5 microns. In particular embodiments, the zirconium powder particles are spherical.

In some embodiments, the oxide layer may be formed during production of the zirconium powder. For example, zirconium powder may be made by the plasma rotating electrode process, and instead of an inert atmosphere in the plasma chamber, controlled amounts of oxygen could be introduced.

In some embodiments, the oxide layer may be formed after the production of the zirconium powder. For example, the oxide layer may be oxides using methods disclosed in U.S. Pat. No. 5,037,438, incorporated herein by reference. However, instead of oxidizing a prosthesis substrate, pure zirconium metal particles or zirconium alloy particles are oxidized. The process conditions include, for instance, air oxidation at elevated temperatures, steam oxidation, water oxidation or oxidation in a salt bath. In some embodiments, the pure zirconium metal particles or zirconium alloy particles are suspended during the oxidation process, for example, by forming a fluidized bed of the particles or by spraying the particles into an oxidizing environment.

Oxidized zirconium powder produced by these or other methods may be used in an additive manufacturing machine and used to produce medical implants such as, but not limited to artificial knee and hip components, as well as components for shoulder implants, ankle implants, and spinal implants, among others.

As noted above, additive manufacturing techniques include those known in the art such as solid free-form fabrication (SFF), selective laser sintering (SLS), direct metal fabrication (DMF), direct metal laser sintering (DMLS), electron beam melting (EBM), and selective laser melting (SLM), among others.

In various embodiments of the present disclosure, additive manufacturing methods allow for three-dimensional structures to be constructed one layer at a time from zirconium powder comprising (a) pure zirconium powder particles with an oxide layer ranging from 0.05 to 5 microns in thickness and/or (b) zirconium alloy powder particles with an oxide layer ranging from 0.05 to 5 microns in thickness. In some of embodiments, the zirconium powder may further comprise pure zirconium powder particles and/or zirconium alloy powder particles that do not contain an oxide layer, other than a native oxide layer that may be present on such particles.

The zirconium powder is solidified by irradiating a layer of the zirconium powder with an energy source such as a laser or an electron beam. The zirconium powder may be selectively melted in some regions, thereby forming substantially nonporous regions. In other regions, the zirconium powder may be incompletely fused to form porous regions. Such substantially nonporous regions and porous regions can be formed by the application of energy from the energy source, which may be directed in raster-scan fashion to selected portions of the zirconium powder layer to melt, fuse and/or sinter the zirconium powder. After forming a pattern in one zirconium powder layer, an additional layer of zirconium powder is dispensed, and the process is repeated until the desired structure is complete.

The desired structures can be formed directly from computer controlled databases, which greatly reduces the time and expense required to fabricate various implants or implant components. For example, a computer-aided system may be employed that has an energy source such as a laser beam or an electron beam to melt, fuse and/or sinter zirconium powder to build the structure one layer at a time according to a model selected in a database of the computer component of the system. In such additive fabrication systems, implants or implant components are formed by sequential delivery of zirconium powder and/or energy to specified points in space to produce the implant or implant component. More particularly, implants or implant components can be produced in a layer-wise fashion from zirconium powder that is dispensed one layer at a time, allowing for the direct manufacture of 3-D structures of high resolution and dimensional accuracy.

In some embodiments, an initial zirconium powder layer may be placed onto a build plate. Thereafter, multiple layers of zirconium powder may be melted, fused and/or sintered due to application of energy from the energy source until the desired structure is complete. In some embodiments, the build plate may form a part of the implant that is implanted into the patient. In some embodiments, the build plate may be removed from the implant component, for example, using a suitable machining process

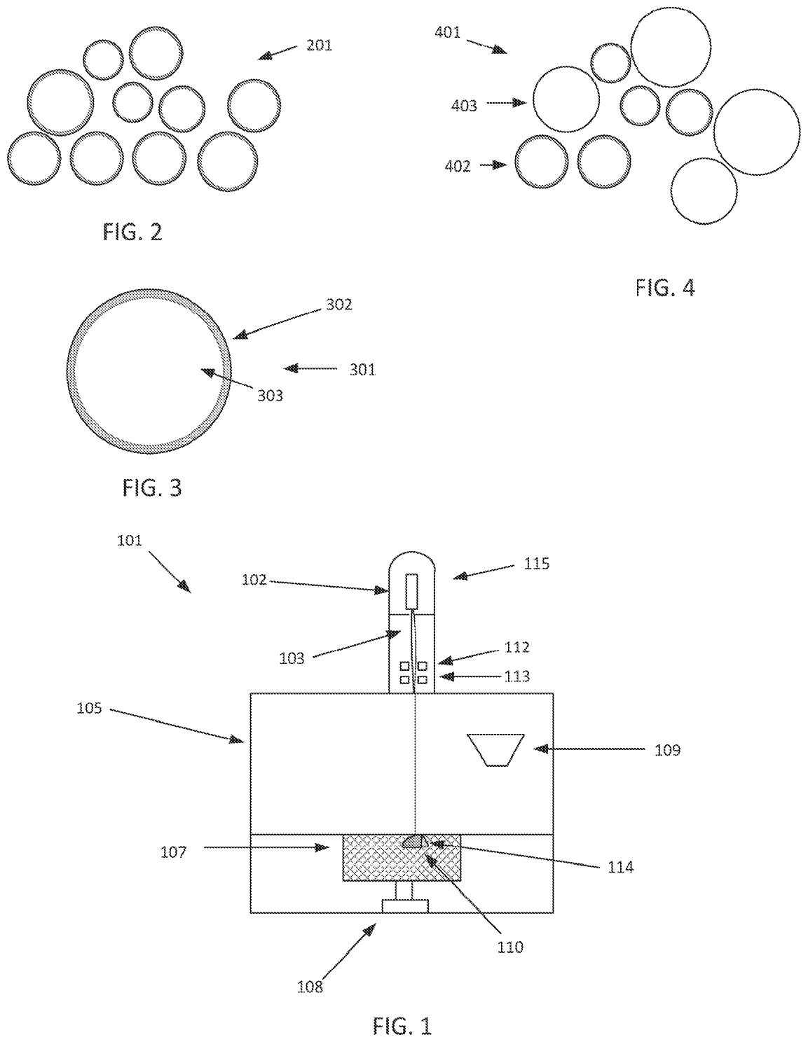

FIG. 1 illustrates an e-beam machine 101 that can be used to additively manufacture medical implants or components using the zirconium powder of the present disclosure. Although an e-beam machine is shown, the zirconium powder of the present disclosure can be used with other additive manufacturing machines, including laser-beam-based machines as described above. An electron beam generator 102 produces electron beam 103 in beam chamber 115. The electrons accelerate through focus coils 112 and direction coils 113. Coils 112 and 113 serve to focus and direct electron beam 103 onto particle bed 107. Particle bed 107 consists of a zirconium powder as described herein, As electron beam 103 impacts the particle bed 107, it causes localized melting, fusing and/or sintering of the zirconium powder. Once the beam has selectively melted, fused and/or sintered portions of the zirconium powder in a prescribed manner, particle bed 107 is lowered by support 108 and powder hopper 109 adds zirconium powder to the build chamber 105. A recoating arm (not shown) by be used to wipe the top of the powder bed to provide a fresh layer for melting. As the process is repeated, implant 110 is produced in successive layers from the melted zirconium powder.

Build chamber 105 typically operates in a vacuum and at elevated temperatures, but usually between 500 degrees F. and 1300 degrees F. A common side effect of e-beam additive manufacturing is the formation of a cake 114 proximate to part 110. The presence of cake 114 is a challenge for e-beam manufacturing as the metal powder needs to be sieved and reused after a build is complete. Caking complicates reuse of the powder. It is believed that the zirconium powder of the present disclosure will substantially prevent caking in e-beam machines.

FIG. 2 illustrates a close up of particles 201 of the powder bed 107 from FIG. 1. Particles 201 may vary somewhat in size and shape, but are typically spherical and may have a particle size distribution with diameters from 5 to 125 microns, more typically from 25-125 microns. Those having ordinary skill in the art will understand that other shapes and sizes could readily be used. It is believed that at the higher temperatures encountered in an e-beam process, pure zirconium powder particles and zirconium alloy powder particles that do not contain an oxide layer (other than a native oxide layer that may be present on such powder particles) that are close to the melt pool will tend to bond and produce caking near the part. However, pure zirconium powder particles and/or zirconium alloy powder particles with an oxide layer as described herein and as schematically illustrated in FIG. 2 are believed to not bond nearly as readily, tending to not cake.

FIG. 3 is a close up of one of the powder particles of FIG. 2. Particle 301 has a pure zirconium or zirconium alloy substrate 303 and a 0.05 to 5 micron oxide layer 302 on the surface of the particle. The oxide layer 302 provides a barrier layers to prevent further oxygen from reacting with the zirconium. As soon as initial pure zirconium powder particles or zirconium alloy powder particles have been oxidized, there is a significantly reduced chance of further oxygen pickup by the powder particles during storage, transportation and use in an additive manufacturing machine. This may increase the stability of the powder in an additive manufacturing process, including enhanced combustion resistance. The oxide layer can be made along the lines described in U.S. Pat. No. 5,037,438, previously incorporated by reference. Alternatively, the oxide layer 302 may be added during production of the zirconium powder. For example, pure zirconium powder particles or zirconium alloy powder particles may be made by the plasma rotating electrode process, and instead of an inert atmosphere in the plasma chamber, controlled amounts of oxygen could be introduced. In some embodiments, the oxide layer may be diffused into the particle substrate.

Because the oxide layer is relatively thin, the properties of the oxidized powder particles 301 should not differ appreciably from a pure zirconium powder, and the additive manufacturing machine operating parameters may not differ appreciably from when a pure powder is used.

Moreover, to the extent that the small amount of oxide introduced by the use of oxidized particles will increase the overall oxygen content of the additive manufacturing part being built, which higher oxygen content may affect the mechanical properties of the part, such impact can be mitigated through design considerations or alternatively by mixing non-oxidized coarser powder with the oxidized zirconium powder described herein.

FIG. 4 illustrates particles of a powder bed 401 that may be used with an additive manufacturing machine of the present disclosure to offset or compensate for any mechanical property changes when producing an implant with an increased oxygen content. In addition to oxidized zirconium powder particles 402, controlled quantities of non-oxidized zirconium powder particles such as non-oxidized pure zirconium powder particles and/or non-oxidized zirconium alloy powder particles 403 may be mixed with the oxidized zirconium powder particles 402.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the invention should not be limited by any of the above-described exemplary embodiments, but should be defined only in accordance with the claims and their equivalents.

While the present disclosure refers to certain embodiments, numerous modifications, alterations, and changes to the described embodiments are possible without departing from the sphere and scope of the present disclosure, as defined in the appended claim(s). For example, while the present disclosure focuses on particles that have been subject to oxidation, similar to oxidation, pure zirconium and zirconium alloy surfaces may be carburized or nitrided to provide the benefits of the present disclosure.

Accordingly, it is intended that the present disclosure not be limited to the described embodiments, but that it has the full scope defined by the language of the following claims, and equivalents thereof. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed, and that the appended claims are intended to be construed to include such variations, except as limited by the prior art.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counterclockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular embodiments. Such terms are not generally limiting to the scope of the claims made herein. Any embodiment or feature of any section, portion, or any other component shown or particularly described in relation to various embodiments of similar sections, portions, or components herein may be interchangeably applied to any other similar embodiment or feature shown or described herein.

While the present disclosure refers to certain embodiments, numerous modifications, alterations, and changes to the described embodiments are possible without departing from the sphere and scope of the present disclosure, as defined in the appended claim(s). Accordingly, it is intended that the present disclosure not be limited to the described embodiments. Rather these embodiments should be considered as illustrative and not restrictive in character. All changes and modifications that come within the spirit of the invention are to be considered within the scope of the disclosure. The present disclosure should be given the full scope defined by the language of the following claims, and equivalents thereof. Unless otherwise defined, all technical terms used herein have the same meaning as commonly understood by one of ordinary skill in the art to which the disclosure belongs.

The foregoing description has broad application. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed, and that the appended claims are intended to be construed to include such variations, except as limited by the prior art.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one embodiment” of the present disclosure are not intended to be interpreted as excluding the existence of additional embodiments that also incorporate the recited features.

In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with another embodiment to yield a still further embodiment. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one”, “one or more”, and “and/or”, as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more embodiments or configurations for the purpose of streamlining the disclosure. However, it should be understood that various features of the certain embodiments or configurations of the disclosure may be combined in alternate embodiments or configurations. Moreover, the following claims are hereby incorporated into this Detailed Description by this reference, with each claim standing on its own as a separate embodiment of the present disclosure.

1. A method of producing a medical implant or a medical implant component by a process that comprises selectively applying energy to zirconium powder particles to build the medical implant or the medical implant component, wherein the zirconium powder particles comprise pure zirconium powder particles with an oxide layer ranging from 0.05 to 5 microns in thickness and/or zirconium alloy powder particles with an oxide layer ranging from 0.05 to 5 microns in thickness.

2. The method of claim 1, wherein the zirconium powder particles are spherical particles.

3. The method of claim 1, wherein at least 95 wt % of the zirconium powder particles range from 5 microns to 125 microns in diameter and/or wherein the zirconium powder particles have a median particle size ranging from 25 to 70 microns in diameter.

4. The method of claim 1, wherein the zirconium powder particles further comprise unoxidized pure zirconium powder particles and/or unoxidized zirconium alloy powder particles.

5. The method of claim 4, wherein an (a):(b) weight ratio of (a) the pure zirconium powder particles with an oxide layer ranging from 0.05 to 5 microns in thickness and/or that zirconium alloy powder particles with an oxide layer ranging from 0.05 to 5 microns in thickness to (b) the unoxidized pure zirconium powder particles and/or the unoxidized zirconium alloy powder particles ranges from 0.5:1 to 2:1.

6. The method of claim 1, wherein the medical implant or medical implant component is produced in a layer-wise fashion by dispensing and irradiating the zirconium powder particles in preselected areas one layer at a time.

7. The method of claim 1, wherein the method comprises repeatedly forming a layer of zirconium powder particles and irradiating the layer of zirconium powder particles with an energy source to melt, fuse and/or sinter the zirconium powder particles until the medical implant or medical implant component is formed.

8. The method of claim 1, wherein the energy is applied by irradiating the zirconium powder particles in predetermined areas with a laser beam or an electron beam

9. The method of claim 1, wherein the energy is applied to the zirconium powder particles in a vacuum and at temperatures ranging from 500 degrees F. to 1300 degrees F.

10. The method of claim 1, wherein the method further comprises oxidizing at least one surface of the medical implant or medical implant component that is formed to create a wear-resistant ceramic surface.

11. A medical implant or medical implant component formed from the method of claim 1.

12. The medical implant or the medical implant component of claim 11, wherein the medical implant or the medical implant component is selected from a hip implant, a knee implant, a shoulder implant, an ankle implant, a spinal implant, a component of a hip implant, a component of a knee implant, a component of a shoulder implant, a component of an ankle implant, or a component of a spinal implant.

13. Zirconium powder particles comprising pure zirconium powder particles with an oxide layer ranging from 0.05 to 5 microns in thickness and/or zirconium alloy powder particles with an oxide layer ranging from 0.05 to 5 microns in thickness.

14. The zirconium powder particles of claim 13, wherein at least 95 wt % of the zirconium powder particles range from 5 microns to 125 microns in diameter and/or wherein the zirconium powder particles have a median particle size ranging from 25 to 70 microns in diameter.

15. The zirconium powder particles of claim 13, wherein the zirconium powder particles comprises spherical particles.
